(19) United States
(12) Patent Application Publication
(10) Pub. No.: US 2012/0302825 A1
(43) Pub. Date: Nov. 29, 2012

(54) Methods of Treating Tissue within a Bodily Passage

(75) Inventors: Darin Schaeffer, Bloomington, IN (US); Thomas Cherry, Covington, LA (US)

(73) Assignee: COOK MEDICAL TECHNOLOGIES LLC, Bloomington, IN (US)

(21) Appl. No.: 13/304,435

(22) Filed: Nov. 25, 2011

Related U.S. Application Data
(60) Provisional application No. 61/417,426, filed on Nov. 28, 2010.
(60) Provisional application No. 61/510,625, filed on Jul. 22, 2011.

(51) Int. Cl. A61M 31/00; A61B 1/04

(52) U.S. Cl. 600/104; 604/514

(57) ABSTRACT

Methods of treatment are described. More particularly, methods of treating a tissue within a bodily passage, such as a sinus cavity, airway, or sinus passage with a catheter comprising an elongate tubular member and one or more balloons. The catheter can comprise a light source and/or camera to assist physicians is navigating the catheter to a point of treatment.

### CROSS-REFERENCE TO RELATED APPLICATIONS

This application claims priority to U.S. Provisional Application No. 61/417,426 filed on Nov. 28, 2010 and U.S. Provisional Application No. 61/510,625 filed on Jul. 22, 2011. The disclosure of each of these related applications is hereby incorporated into this disclosure in its entirety.

### FIELD

The invention relates generally to methods of treatment. More particularly, the invention relates to methods of treating tissue in a bodily passage, such as a sinus cavity or airway.

### BACKGROUND

It is sometimes necessary or otherwise desirable to medicate a point of treatment in a bodily passage to reduce inflammation or treat an infected area. For example, to treat chronic sinusitis or eustachian tube dysfunction, physicians sometimes systematically administer drugs at a treatment site within the sinus. If the administration of drugs is determined to be unsuccessful at treating the sinusitis, surgery may be required and physicians can use a variety of techniques for treatment, such as by removing affected tissue, dilating the affected area using a balloon catheter, or a combination of these two techniques. Subsequent to performing the selected technique, physicians may continue to systematically administer medication to assist with recovery of the treatment site by flushing the treatment site with medication, or having the patient inhale the medication using an inhaler.

In another example, to treat a stricture within an airway, physicians treat the stricture by dilating the affected area using, for example, non-coated balloon catheters. Subsequent to dilating the stricture, physicians systematically administer medication to assist with recovery of the treatment site by applying medication topically using a small piece of gauze, or injecting a medication using a needle.

The systematic administration of drugs prior to, or subsequent to, the performance of a procedure may have negative effects on a patient. For example, the long-term use of corticosteroids has been associated with spasms of the large airways, fungus infection, decreased bone thickness, and/or growth impediments. In addition, advancing multiple devices to a point of treatment subsequent to accomplishing the removal of tissue and/or dilation of a treatment site increases the complexity of the procedure and the amount of time required to complete the procedure.

Therefore, a need exists for methods of treating tissue within a bodily passage, such as a sinus cavity or airway.

### SUMMARY

Several exemplary methods of treating tissue within a bodily passage, such as a sinus cavity, airway, or sinus passage are described herein.

Various methods of treating tissue in a bodily passage using a catheter are described. For example, methods of treating tissue in a sinus cavity using a catheter are described. In another example, methods of treating tissue in an airway using a catheter are described. In another example, methods of treating tissue in a sinus passage using a catheter are described.

Additional understanding of the methods contemplated and/or claimed by the inventor can be gained by reviewing the detailed description of exemplary embodiments, presented below, and the referenced drawings.

### BRIEF DESCRIPTION OF THE DRAWINGS

FIG. 1 is a flowchart representation of an exemplary method of treatment.

FIG. 2 is a flowchart representation of another exemplary method of treatment.

FIG. 3 is a flowchart representation of another exemplary method of treatment.

FIG. 4 is a flowchart representation of another exemplary method of treatment.

FIG. 5 is a flowchart representation of another exemplary method of treatment.

### DETAILED DESCRIPTION

The following detailed description and the appended figures are provided to describe and illustrate exemplary embodiments of the invention for the purpose of enabling one of ordinary skill in the relevant art to make and use the invention. The description and drawings are not intended to limit the scope of the invention, or its protection, in any manner.

As used herein, the terms “proximal” and “distal” are used to describe opposing axial ends of the particular elements or features being described. The term “bodily passage” refers to any passage within the body of an animal, including, but not limited to, humans, and includes elongate passages. The term “medication” refers to any fluid, drug, and/or agent used to treat a patient. The term “sinus passage” refers to the nasal passages and includes, but is not limited to, eustachian tube(s), primary ostium, and/or an accessory ostium. The term “airway” refers to any airway including, but not limited to, the nasopharynx, oropharynx, pharynx, trachea, bronchial tubes, esophagus, and/or lungs. The term “sinus cavity” refers to the frontal, ethmoid, sphenoid, and/or maxillary sinus. The term “damage” refers to shattering, cracking, breaking, fracturing, fragmenting, puncturing, penetrating, ripping, tearing, and/or perforating.

Various methods of treatment are provided. These methods include treating a tissue in a bodily passage, such as a sinus cavity, airway, or sinus passage. While, the methods of treating tissue are exemplified by methods of treating tissue in a sinus cavity, airway, or sinus passage, the methods can also be used to treat other bodily passages.

While the methods described herein are shown and described as a series of acts, it is to be understood and appreciated that the methods are not limited by the order of acts, as some acts may, in accordance with these methods, occur in different orders with other acts described herein, and/or concurrently with other acts described herein.

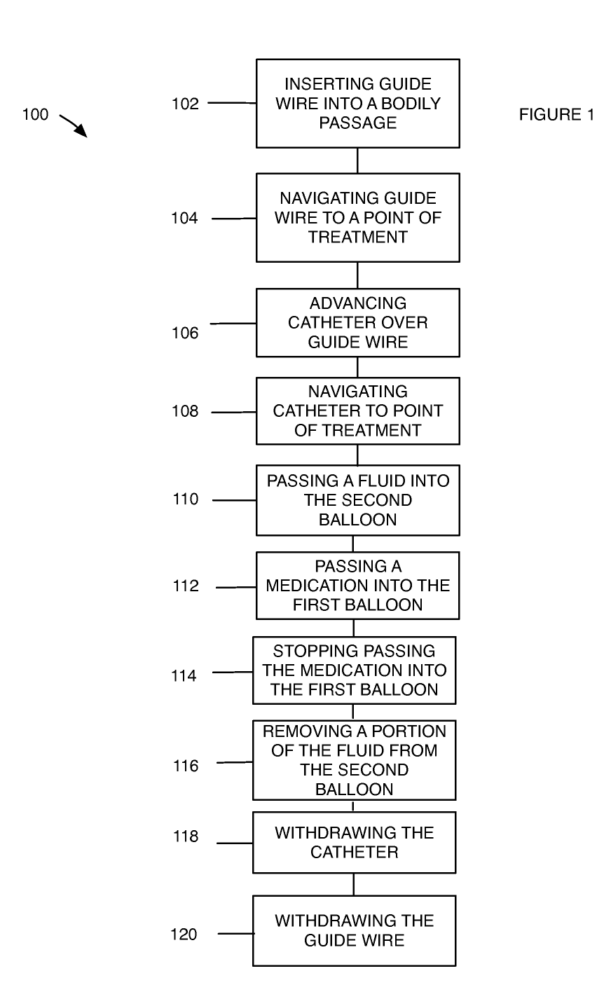

FIG. 1 is a flowchart representation of an exemplary method 100 of treating tissue in a bodily passage defined by a passage wall.

An initial step 102 comprises inserting a guide wire having a proximal end and a distal end into a bodily passage such that the distal end of the guide wire is disposed in the bodily passage. Another step 104 comprises navigating the distal end of the guide wire to a point of treatment within said bodily passage. Another step 106 comprises advancing a catheter having a proximal end and a distal end over the previously placed guide wire such that the distal end of the catheter is disposed in said bodily passage. The catheter comprises an elongate tubular member, a first balloon, and a second balloon. The elongate tubular member has a proximal end, a distal end, an inflation port, and an infusion port and defines an inflation lumen and an infusion lumen. The inflation lumen extends between the inflation port and the distal end of the elongate tubular member. The infusion lumen extends between the infusion port and the distal end of the elongate tubular member. The first balloon is disposed on the distal end of the elongate tubular member and has a wall defining one or more pores extending through the wall of the first balloon that allow fluid (e.g., medication) to pass through the wall of the first balloon. The first balloon is in communication with the infusion lumen. The second balloon is disposed within the first balloon and on the distal end of the elongate tubular member. The second balloon is moveable between a deflated configuration and an inflated configuration and is in communication with the inflation lumen. Another step 108 comprises navigating the distal end of the catheter to a point of treatment within said bodily passage. Another step 110 comprises passing a fluid through the inflation lumen and into the second balloon with a pressure sufficient to inflate the second balloon and provide contact between a portion of the first balloon and the passage wall. Another step 112 comprises passing a medication through the infusion lumen into the first balloon with a pressure sufficient to expel the medication through one or more pores. Another step 114 comprises stopping the step of passing a medication through the infusion lumen and into the first balloon. Another step 116 comprises removing a portion of the fluid from the second balloon. Another step 118 comprises withdrawing the distal end of the catheter from the bodily passage. Another step 120 comprises withdrawing the distal end of the guide wire from the bodily passage.

The step of inserting the distal end of a guide wire into a bodily passage can be accomplished using a guide wire independent of any other device. Alternatively, the guide wire can pass through the working channel of a visualization scope. The guide wire can have any suitable length and be formed of any suitable material. Optionally the guide wire can comprise a distal end with a light source disposed thereon and configured to emit light from the distal end of the guide wire and/or a camera disposed thereon configured to transmit images to a display. A light source and/or camera assist physicians in positioning the distal end of the guide wire at a point of treatment.

The step of navigating the distal end of the guide wire to a point of treatment can be accomplished using direct visualization (e.g., with a scope). Alternatively, when the distal end of the guide wire comprises a light source and/or camera, navigating the distal end of the guide wire to a point of treatment can be accomplished transcutaneously and/or via live feed on a display. In an example, navigating the distal end of the guide wire to a point of treatment using transcutaneous visualization can be accomplished by visualizing the intensity of the light transcutaneously to determine if the intensity is indicative of proper positioning of the distal end of the guide wire at a point of treatment. In another example, navigating the distal end of the guide wire to a point of treatment can be accomplished by viewing images provided on a display and determining if the images indicate proper positioning of the distal end of the guide wire.

The step of advancing the distal end of the catheter over the previously placed guide wire can be accomplished by inserting the proximal end of the guide wire through a lumen defined by the elongate tubular member and advancing the catheter distally over the previously placed guide wire. Alternatively, the steps of inserting the distal end of a guide wire into a bodily passage and navigating the distal end of the guide wire to a point of treatment can be omitted and the distal end of the catheter can be inserted into the bodily passage independently.

The catheter can comprise any suitable structure for accomplishing the methods described herein. Exemplary catheter structures are described below, but are not intended to be limiting in nature.

An exemplary catheter comprises an elongate tubular member, a first balloon, and a second balloon. The elongate tubular member extends between a proximal end and a distal end, has an inflation port, and an infusion port and defines an inflation lumen and an infusion lumen. Optionally, the elongate tubular member can include a guide wire lumen. The infusion lumen extends between the infusion port and a first opening positioned on the distal end of the elongate tubular member. The inflation lumen extends between the inflation port and a second opening positioned on the distal end of the elongate tubular member.

The first balloon and second balloon are disposed on the distal end of the elongate tubular member; the second balloon being disposed within the first balloon. The material of the first balloon, the portion of the exterior surface of the elongate tubular member positioned within the first balloon, and the exterior surface of the second balloon define an infusion chamber. The first balloon is disposed on and positioned on the distal end of the elongate tubular member such that the opening of the infusion lumen is in communication with the infusion chamber. The material of the second balloon and the portion of the exterior surface of the elongate tubular member positioned within the second balloon define an inflation chamber. The second balloon is disposed on and positioned on the distal end of the elongate tubular member such that the second opening of the inflation lumen is in communication with the inflation chamber.

The second balloon is adapted to move between deflated and inflated configurations as fluid is moved into and out of the inflation chamber via the inflation port, inflation lumen and the first opening of the inflation lumen. A user inflates the second balloon by introducing an appropriate fluid, such as saline, into the inflation port and through the inflation lumen until the fluid passes through the first opening of the inflation lumen into the inflation chamber. The resulting pressure placed on the inner surface of the second balloon by the fluid causes the second balloon to inflate and adopt the inflated configuration. To move the second balloon to the deflated configuration, vacuum pressure can be applied to the inflation port to remove fluid located within the inflation chamber, resulting in the balloon deflating.

Similar to the second balloon, the first balloon is adapted to move between deflated and inflated configurations by way of movement of the second balloon between its inflated and deflated configurations. Thus, the user expands the first balloon by inflating the second balloon. To move the first balloon to the deflated configuration, vacuum pressure can be applied to the inflation port to remove fluid within the inflation chamber resulting in the second balloon returning to its deflated configuration.

The first and second balloons advantageously include memory imparted onto the balloons by a heat treatment step that comprises heating the balloons while they are in their deflated configurations. This heat treatment, and the resulting memory, gives the balloons a tendency to return to their deflated configurations. Alternatively, the heat treatment step can be omitted or applied to one of the first balloon or second balloon independently. For example, when only the first balloon has been heat treated, the first balloon will have a tendency to return to its deflated configuration when the second balloon is moved from its inflated configuration to its deflated configuration.

The first balloon includes one or more regions that include one or more pores. Each of the pores extends through the material of the first balloon and permit fluid (e.g., medication) to pass through the wall of the first balloon, preferably with the application of pressure within the infusion chamber. Examples of suitable diameters for each of the one or more pores include diameters in the range from about 0.01 mm to about 0.25 mm. Additional exemplary diameters considered suitable for each of the one or more pores include diameters in the range from about 0.025 mm to about 0.15 mm. Further exemplary diameters considered suitable for each of the one or more pores include diameters in the range from about 0.05 mm to about 0.1 mm. Alternatively, when at least two or more pores are provided, at least two of the pores can vary in diameter. For example, the pores can increase in diameter from the proximal end to the distal end of the first balloon. In a further alternative, when at least two or more pores are provided, the pores can increase in number from the proximal end to the distal end of the first balloon. The variable size and number configurations advantageously provide substantially equalized distribution and/or a reduced pressure drop of the medication as it is being passed through the wall of the first balloon from the proximal end to the distal end of the first balloon.

The regions can be arranged on the first balloon in any suitable configuration. For example, the regions can circumferentially-extend around the first balloon. Other contemplated configurations include a single circumferentiallyextending region, multiple circumferentially-extending regions, a staggered configuration, and/or a linear configuration along the length of the first balloon.

Depending on the size and number of pores defined by the wall of the first balloon, the first balloon can be adapted to move between a deflated and inflated configuration as a medication is moved into and out of the infusion chamber via the infusion port, infusion lumen and the second opening of the infusion lumen. Configurations that allow for the first balloon to move between a deflated and inflated configuration advantageously provide two different inflated configurations for the treatment catheter. For example, a first configuration where the second balloon is inflated and a second configuration where the first balloon is inflated in combination with the second balloon.

While the catheter has been described as including two balloons, a first outer balloon and a second inner balloon, the catheter can alternatively comprise a single balloon defining the one or more regions containing one or more pores as described above. When a single balloon is disposed on the distal end of the elongate tubular member, the elongate tubular member has an infusion port and defines an infusion lumen with an opening on the distal end of the elongate tubular member. Optionally, the elongate tubular member can comprise a light source configured to emit light from the catheter and/or a camera configured to transmit images to a display, each of which can be disposed within the opening defined by the elongate tubular member. The single balloon is disposed on to the distal end of the elongate tubular member and the material of the single balloon and the portion of the exterior surface of the elongate tubular member positioned within the single balloon define an infusion chamber. The single balloon is positioned on the distal end of the elongate tubular member such that the opening of the infusion lumen is in communication with the infusion chamber. The single balloon is adapted to move between deflated and inflated configurations as a medication is moved into and out of the infusion chamber via the infusion port, infusion lumen and the first opening of the infusion lumen. The amount of inflation of the single balloon will depend on the number and diameter of the one or more pores defined by the wall of the single balloon and the amount, and rate, of medication being introduced into the infusion chamber.

Additional structure can be attached to the catheters described herein to facilitate the inflation and deflation of the balloon(s). For example, a syringe or other suitable structure can be attached to the inflation port and/or infusion port using any suitable connection, such as a luer lock connection. The fluid and/or medication can be stored within the syringe, inflation lumen, and/or infusion lumen and can be introduced into and removed from the inflation and/or infusion chamber by operating the syringe using conventional practices.

Another exemplary catheter considered suitable to accomplish the herein described methods is described below and is similar to the dual catheter described above, except as indicated. The catheter is adapted to be used with a light source and comprises an elongate tubular member, a first balloon, a second balloon, and a fiber optic. The elongate tubular member comprises a proximal end, a distal end, and defines an inflation lumen, an infusion lumen, and an optic lumen. The first balloon comprises a proximal portion, a distal portion, and an intermediate portion disposed between the proximal and distal portions. The intermediate portion defines the maximum diameter of the first balloon when the second balloon is in the inflated configuration. The fiber optic is disposed in the optic lumen and is adapted to be operatively connected to a light source. The first and second balloon and/or the fiber optic are adapted to emit light radially along the intermediate portion of the first balloon and/or distally from the distal end of the elongate tubular member.

The above-described catheter configurations, and variations thereof, are described in greater detail in co-pending application titled “Catheters and Methods for Identification and Treatment of Bodily Passages,” filed concurrently with the present application, having application Ser. No. 13/304,432, and hereby incorporated by reference into this disclosure in its entirety. Exemplary support describing structures considered suitable to perform the herein described methods are found in FIGS. 1 through 3C, and paragraphs [0024] through [0064].

While the steps of navigating the distal end of the guide wire to a point of treatment within said bodily passage and advancing a catheter having a proximal end and a distal end over the previously placed guide wire such that the distal end of the catheter is disposed in said bodily passage have been described as separate steps, these steps can be accomplished concurrently. In an alternative, the step of advancing a catheter having a proximal end and a distal end over the previously placed guide wire such that the distal end of the catheter is disposed in said bodily passage can be accomplished prior to the step navigating the distal end of the guide wire to a point of treatment within said bodily passage.

The step of navigating the distal end of the catheter to a point of treatment within said bodily passage can be accomplished using direct visualization of the distal end of the catheter (e.g., with a scope). Optionally, the distal end of the catheter can include a light source configured to emit light from the catheter and/or a camera configured to transmit images to a display. A light source and/or camera assist physicians in positioning the distal end of the catheter at a point of treatment. When the distal end of the catheter includes a light source and/or camera, navigating the distal end of the catheter to a point of treatment can be accomplished transcutaneously and/or via live feed on a display, as described above.

While the steps of navigating the distal end of the guide wire to a point of treatment within said bodily passage and navigating the distal end of the catheter to a point of treatment within said bodily passage have been described as separate steps, these steps can be accomplished concurrently. In addition, while the step of navigating the distal end of the guide wire to a point of treatment has been described, the distal end of the guide wire can alternatively be navigated proximal to, or distal to, the point of treatment. Furthermore, while the step of navigating the distal end of the catheter to a point of treatment has been described, the distal end of the catheter can alternatively be navigated proximal to, or distal to, the point of treatment.

Alternatively, the steps of advancing the distal end of the catheter into a bodily passage and navigating the distal end of the catheter to a point of treatment can be accomplished using a guiding catheter, with or without the use of a guide wire. When a guiding catheter is used in combination with a guide wire, an initial step comprises inserting the distal end of a guiding catheter into a bodily passage of a patient. Another step comprises navigating the distal end of the guiding catheter to a point of treatment using direct visualization. Another step comprises inserting the distal end of a guide wire into a lumen of the guiding catheter. Another step comprises navigating the distal end of the guide wire to a point of treatment. Another step comprises advancing the distal end of a catheter over the guide wire. Another step comprises navigating the distal end of the catheter over the guide wire to a point of treatment.

The guiding catheter can optionally include a light source configured to emit light from the guiding catheter and/or a camera configured to transmit images to a display. A light source and/or camera assist physicians in positioning the distal end of the guiding catheter at a point of treatment. When the distal end of the guiding catheter includes a light source and/or camera, navigating the guiding catheter to a point of treatment can be accomplished transcutaneously and/or via live feed on a display, as described above.

In a further alternative, the steps of advancing the distal end of the catheter into a bodily passage and navigating the distal end of the catheter to a desired point of treatment can be accomplished using a guiding catheter independent of a guide wire. When a guiding catheter is used independent of a guide wire, an initial step comprises inserting the distal end of a guiding catheter into a bodily passage of a patient. Another step comprises navigating the distal end of the guiding catheter to a point of treatment via direct visualization, transcutaneously, and/or live feed on a monitor. Another step comprises inserting the distal end of a catheter through a lumen of the guiding catheter. Another step comprises navigating the distal end of the catheter through the lumen of the guiding catheter to a point of treatment.

The step of passing a fluid through the inflation lumen and into the second balloon with a pressure sufficient to inflate the second balloon and provide contact between a portion of, the entirety of, or a majority of, the first balloon and the passage wall can be accomplished by introducing a fluid into the inflation port to advance the second balloon from its deflated to its inflated configuration. The amount of the exterior surface of the first balloon contacting the passage wall, and the amount of pressure exerted by the exterior surface of the first balloon onto the passage wall, will depend on the amount fluid being passed into the inflation chamber of the second balloon.

For example, in procedures where a large amount of contact and pressure are desired between the exterior surface of the first balloon and the passage wall, a larger amount of fluid will be passed through the inflation port into the inflation chamber. In another example, in procedures where a smaller amount of contact and pressure are desired between the exterior surface of the first balloon and the passage wall, a smaller amount of fluid will be passed through the inflation port into the inflation chamber. Examples of pressures considered suitable include pressures in the range from about 1 ATM to about 12 ATM.

Examples of fluids considered suitable to pass through the inflation lumen into the second balloon include saline, water, contrast, or a mixture of one or more of saline, water, and/or contrast.

The step of passing a medication through the infusion lumen into the first balloon with a pressure sufficient to expel the medication through the one or more pores can be accomplished by passing a medication through the infusion port and infusion lumen. This step can be accomplished prior, during, or subsequent to passing a fluid into the inflation chamber. The medication can be passed into the infusion lumen using any suitable device (e.g., a syringe in communication with the infusion lumen). By inflating the second balloon to a pressure suitable to provide contact between the exterior surface of the first balloon and the passage wall, the medication being expelled by the one or more pores can advantageously be infused into the passage wall defining the bodily passage. It is considered advantageous to pass medication through the infusion lumen and into the first balloon while the second balloon is in its inflated configuration to infuse the medication within the passage wall.

For example, in procedures where it is desired to infuse a larger amount of medication at a point of treatment, a larger amount of fluid will be passed through the inflation port into the inflation chamber. Thus, providing a larger amount of contact and pressure between the exterior surface of the first balloon and the passage wall. In another example, in procedures where it is desired to infuse a smaller amount of medication at a point of treatment, a smaller amount of fluid will be passed through the inflation portion into the inflation chamber. Thus, providing a smaller amount of contact and pressure between the exterior surface of the first balloon and the passage wall.

Examples of pressures considered suitable to expel the medication through the one or more pores and infuse the medication into the wall defining the bodily passage include pressures in the range from about 1 ATM to about 10 ATM. Skilled artisans will appreciate however, that the pressure required to infuse the medication will correlate with the inflated pressure of the first balloon.

The medication can comprise any suitable fluid, drug, and/or agent used to treat a patient. Examples of medications considered suitable include, but are not limited to, anti-inflammatories (e.g., steroids), antineoplastics (e.g., mitomycin), cytotoxics, adrenaline (e.g., epinephrine), antibiotics, antifungal agents, and/or anti-proliferatives (e.g., such as those used on coronary stents), or a combination thereof.

While the steps of navigating the distal end of the catheter to a point of treatment within said bodily passage, passing a fluid through the inflation lumen and into the second balloon with a pressure sufficient to inflate the second balloon, and provide contact between a portion of the first balloon and the passage wall, and passing a medication through the infusion lumen into the first balloon with a pressure sufficient to expel the medication through one or more pores have been described as separate steps, these steps can be accomplished concurrently. In another alternative, the step of navigating the distal end of the catheter to a point of treatment within said bodily passage can be accomplished concurrently with the step of passing a fluid through the inflation lumen and into the second balloon with a pressure sufficient to inflate the second balloon and provide contact between a portion of the first balloon and the passage wall. In another alternative, the step of navigating the distal end of the catheter to a point of treatment within said bodily passage can be accomplished concurrently with passing a medication through the infusion lumen into the first balloon, or single balloon, with a pressure sufficient to expel the medication through one or more pores. In a further alternative, the step of passing a medication through the infusion lumen into the first balloon with a pressure sufficient to expel the medication through one or more pores can be accomplished prior to or concurrently with the step of passing a fluid through the inflation lumen and into the second balloon with a pressure sufficient to inflate the second balloon and provide contact between a portion of the first balloon and the passage wall and/or the step of navigating the distal end of the catheter to a point of treatment within said bodily passage.

The step of stopping the step of passing a medication through the infusion lumen and into the first balloon can be accomplished by terminating the passing of the medication through the infusion lumen. In an example where a syringe is the device passing the medication into the infusion lumen, this can be accomplished by the physician removing pressure from the plunger of the syringe.

The step of removing a portion of the fluid from the second balloon can be accomplished by removing the fluid passed into the inflation chamber. For example, a syringe in communication with the inflation port can be used to provide vacuum pressure to remove the fluid from the inflation chamber. The amount of fluid removed from the inflation chamber can vary depending on the procedure. For example, alternative to removing a portion of the fluid, all of the fluid, or as much as possible, can be removed from the inflation chamber.

While the steps of stopping the step of passing a medication through the infusion lumen and into the first balloon and removing a portion of the fluid from the second balloon have been described as separate steps, these steps can be accomplished concurrently. In another alternative, the step of stopping the step of passing a medication through the infusion lumen and into the first balloon can be accomplished prior to the step of removing a portion of the fluid from the second balloon.

The step of withdrawing the distal end of the catheter from the bodily passage can be accomplished by pulling the catheter proximally over the guide wire until it is completely removed from the bodily passage and the patient. Alternatively, if the distal end of the catheter has been navigated to a point of treatment independent of a guide wire, the step of withdrawing the distal end of the catheter from the bodily passage can be accomplished by pulling the catheter proximally until the distal end of the catheter is completely removed from the bodily passage and the patient.

While the steps of passing a medication through the infusion lumen into the first balloon with a pressure sufficient to expel the medication through one or more pores, removing a portion of the fluid from the second balloon, and withdrawing the distal end of the catheter from the bodily passage have been described as separate steps, these steps can be accomplished concurrently. In another alternative, the steps of passing a medication through the infusion lumen into the first balloon with a pressure sufficient to expel the medication through one or more pores and removing a portion of the fluid from the second balloon can be accomplished concurrently and the step of withdrawing the distal end of the catheter can subsequently be accomplished. In another alternative, the step of passing a medication through the infusion lumen into the first balloon with a pressure sufficient to expel the medication through one or more pores can be accomplished and the steps of removing a portion of the fluid from the second balloon and withdrawing the distal end of the catheter from the bodily passage can be accomplished subsequently and concurrently.

While the steps of stopping the step of passing a medication through the infusion lumen and into the first balloon, removing a portion of the fluid from the second balloon, and withdrawing the distal end of the catheter from the bodily passage have been described as separate steps, these steps can be accomplished concurrently. In another alternative, the step of stopping the step of passing a medication through the infusion lumen and into the first balloon can be accomplished and the steps removing a portion of the fluid from the second balloon and withdrawing the distal end of the catheter from the bodily passage can be accomplished concurrently.

The step of withdrawing the distal end of the guide wire from the bodily passage can be accomplished by pulling the guide wire proximally until it is completely removed from the bodily passage and the patient. Alternatively, this step can be accomplished in combination with drawing the catheter from the bodily passage.

If a guiding catheter has been used to advance the catheter to a point of treatment, a required additional step of withdrawing the distal end of the guiding catheter from the bodily passage is accomplished concurrently with drawing the distal end of the catheter and/or guide wire, or subsequent to withdrawing the distal end of the catheter and/or guide wire.

Optional steps of inserting the distal end of a cutting tool into the bodily passage, navigating the distal end of the cutting tool to a point of treatment, cutting tissue from the bodily passage, removing the tissue, and withdrawing the cutting tool can be accomplished prior to advancing the catheter, guiding catheter, and/or guide wire into a bodily passage and to a point of treatment. Any suitable cutting tool can be used to accomplish these steps. For example, the cutting tool can comprise a suction cutter or scalpel. To accomplish these steps, an initial step comprises inserting the distal end of a cutting tool into a bodily passage. Another step comprises navigating the distal end of the cutting tool to a point of treatment. The step of navigating the distal end of a cutting tool to a point of treatment can be accomplished using direct and/or transcutaneous visualization. Another step comprises cutting tissue from the bodily passage. Another step comprises removing the tissue from the bodily passage. Another step comprises removing the distal end of the cutting tool from the bodily passage.

Another optional step comprises inflating the second balloon to a pressure sufficient to damage the passage wall surrounding the exterior surface of the first balloon. This step can be performed after, or replace, the step of passing a fluid through the inflation lumen and into the second balloon with a pressure sufficient to inflate the second balloon and provide contact between a portion of the first balloon and the passage wall. This step can be accomplished by inflating the second balloon to a pressure in the range from about 2 ATM to about 18 ATM. It is considered advantageous to damage the passage wall to provide areas in the passage wall that allow for the infusion of medication passing through the first balloon while the second balloon is in its inflated configuration.

An additional optional step comprises reducing the pressure of the second balloon after damaging the passage wall to a pressure sufficient to provide contact between a portion of the first balloon and the passage wall. This step can be accomplished by removing a portion of the fluid from the inflation chamber until a desired pressure is reached.

While various catheter configurations, steps, alternative steps, and optional steps have been described above with respect to treating tissue in a bodily passage, these catheter configurations, steps, alternative steps, and optional steps can be included in, accomplished concurrently with, and/or accomplished in the alternative to, the methodologies, catheter configurations, steps, alternative steps, and/or optional steps described below with respect to treating tissue in a sinus cavity, airway, and/or sinus passage.

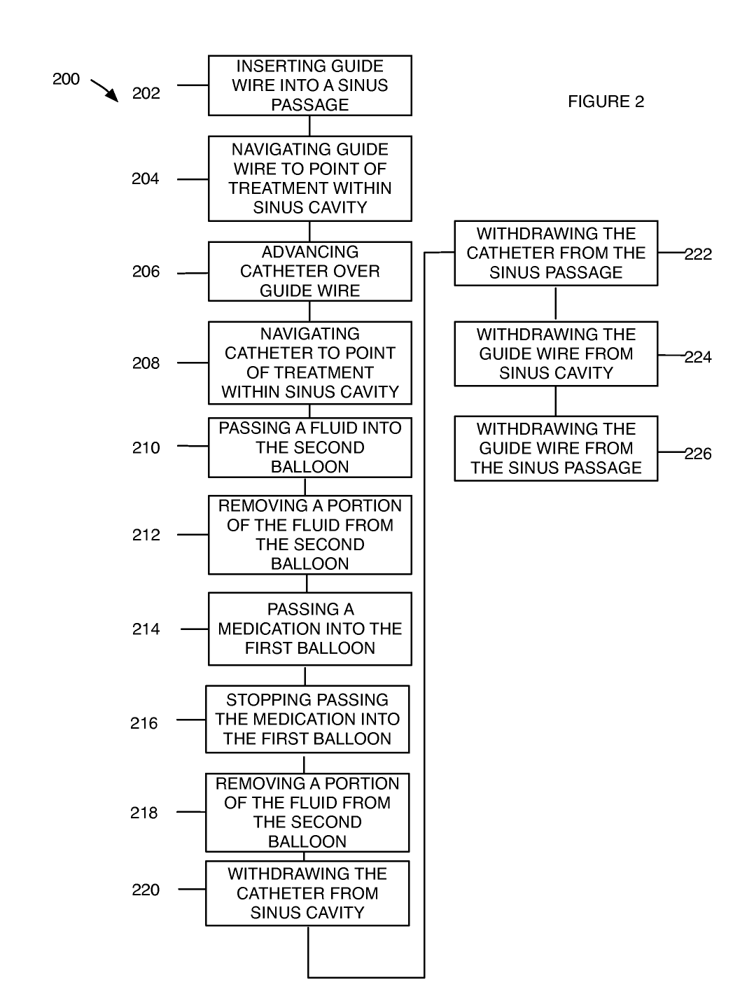

FIG. 2 is a flowchart representation of an exemplary method 200 of treating tissue in a sinus cavity defined by a cavity wall. The method 200 is similar to that described above with respect to method 100, except as described below. An initial step 202 comprises inserting a guide wire having a first proximal end and a first distal end into a sinus passage such that the first distal end of a guide wire is disposed in the sinus passage of a patient. Another step 204 comprises navigating the first distal end of the guide wire to a point of treatment within a sinus cavity. Another step 206 comprises advancing a catheter having a second proximal end and a second distal end over the previously placed guide wire such that the second distal end is disposed in the sinus passage. Another step 208 comprises navigating the second distal end of the catheter to a point of treatment within the sinus cavity. Another step 210 comprises passing a fluid through the inflation lumen and into the second balloon with a pressure sufficient to inflate the second balloon and damage the cavity wall. Another step 212 comprises removing a portion of the fluid from the second balloon to a pressure sufficient to provide contact between a portion of the first balloon and the cavity wall to allow medication to be delivered through the pores of the first balloon. Another step 214 comprises passing a medication through the infusion lumen and into the first balloon with a pressure sufficient to expel the medication through the one or more pores. Another step 216 comprises stopping the step of passing a medication through the infusion lumen and into the first balloon. Another step 218 comprises removing a portion of the fluid from the second balloon. Another step 220 comprises withdrawing the distal end of the catheter from the sinus cavity. Another step 222 comprises withdrawing the distal end of the catheter from the sinus passage. Another step 224 comprises withdrawing the distal end of the guide wire from the sinus cavity. Another step 226 comprises withdrawing the distal end of the guide wire from the sinus passage.

The sinus passage and/or cavity wall can be treated using any of the herein described methods and/or steps by advancing the distal end of one or more of the devices described herein through a primary ostium, an accessory ostium, and/or a ventilation tube disposed in a cavity wall.

The step of inflating the second balloon with a pressure sufficient to damage the cavity wall can be accomplished by inflating the second balloon to a pressure in the range from about 2 ATM to about 18 ATM. It is considered advantageous to damage the passage wall to provide areas in the cavity wall that allow for the infusion of medication passing through the first balloon while the second balloon is in its inflated configuration.

The steps of passing a fluid through the inflation lumen and into the second balloon with a pressure sufficient to inflate the second balloon and damage the cavity wall and removing fluid from the second balloon to a pressure sufficient to provide contact between a portion of the first balloon and the cavity wall are both optional. For example, in an alternative method, the steps of passing a fluid through the inflation lumen and into the second balloon with a pressure sufficient to inflate the second balloon and damage the cavity wall and removing fluid from the second balloon to a pressure sufficient to provide contact between a portion of the first balloon and the cavity wall can be omitted and replaced with a step comprising passing a fluid through the inflation lumen and into the second balloon with a pressure sufficient to inflate the second balloon and provide contact between a portion of the first balloon and the cavity wall. It is considered advantageous to pass medication through the infusion lumen into the first balloon when the second balloon is in its inflated configuration (e.g., damaging the cavity wall and/or contacting the cavity wall) so that the medication can be infused within the cavity wall.

The step of passing fluid through the inflation lumen and into the second balloon with a pressure sufficient to inflate the second balloon and provide contact between a portion of the first balloon and the cavity wall can be accomplished by inflating the second balloon to a pressure in the range from about 1 ATM to about 12 ATM.

In a further alternative, the step of passing a fluid through the inflation lumen and into the second balloon with a pressure sufficient to inflate the second balloon and damage the cavity wall can be accomplished and the step of removing fluid from the second balloon to a pressure sufficient to provide contact between a portion of the first balloon and the cavity wall can be omitted. This advantageously allows for the infusion of medication while the second balloon is inflated to a pressure sufficient to damage the cavity wall.

While the steps of withdrawing the distal end of the catheter from the sinus cavity and withdrawing the distal end of the guide wire from the sinus cavity have been described as separate steps these steps can be accomplished concurrently. In another alternative, the step of withdrawing the distal end of the guide wire from the sinus cavity can be accomplished and the step of withdrawing the distal end of the catheter from the sinus cavity can be subsequently accomplished.

In addition, while the steps of withdrawing the distal end of the catheter from the sinus passage and withdrawing the distal end of the guide wire from the sinus passage have been described as separate steps, these steps can be accomplished concurrently. In another alternative, the step of withdrawing the distal end of the guide wire from the sinus passage can be accomplished and the step of withdrawing the distal end of the catheter from the sinus passage can be subsequently accomplished.

While various catheter configurations, steps, alternative steps, and optional steps have been described above with respect to treating tissue in a sinus cavity, these catheter configurations, steps, alternative steps, and optional steps can be included in, accomplished concurrently with, and/or accomplished in the alternative to, the methodologies, catheter configurations, steps, alternative steps, and/or optional steps described above and/or below with respect to treating tissue in a bodily passage, airway, and/or sinus passage.

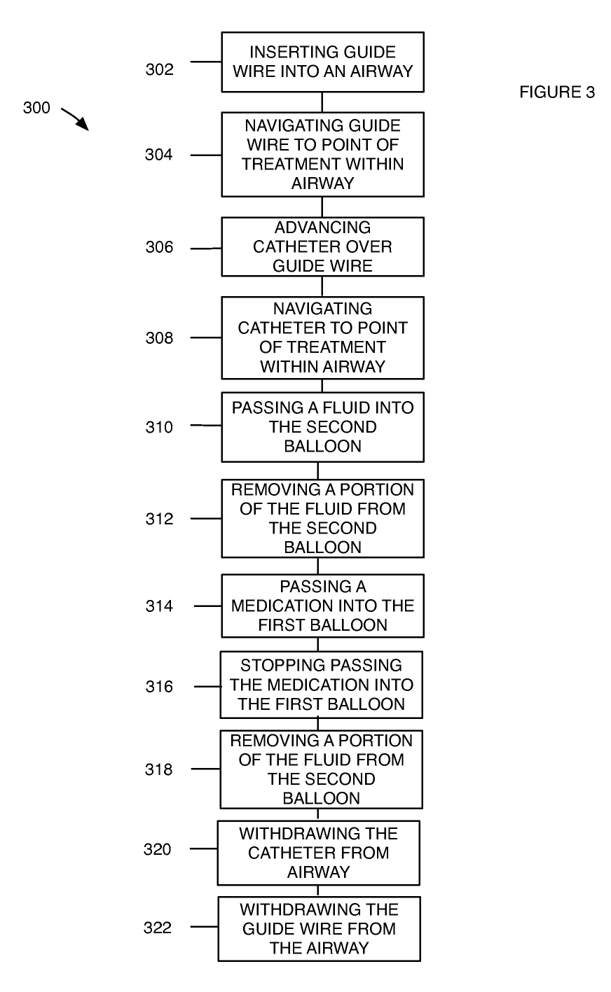

FIG. 3 is a flowchart representation of an exemplary method 300 of treating tissue within an airway defined by an airway wall. The method 300 is similar to that described above with respect to method 100, except as described below. An initial step 302 comprises inserting a guide wire having a first proximal end and a first distal end into an airway such that the first distal end is disposed in the airway. Another step 304 comprises navigating the first distal end of the guide wire to a point of treatment within the airway. Another step 306 comprises advancing a catheter having a second proximal end and a second distal end over the previously placed guide wire such that the second distal end is disposed in the airway. Another step 308 comprises navigating the second distal end of the catheter to a point of treatment within the airway. Another step 310 comprises passing a fluid through the inflation lumen and into the second balloon with a pressure sufficient to inflate the second balloon and dilate the point of treatment within the airway. Another step 312 comprises removing a portion of the fluid within the second balloon to a pressure sufficient to provide contact between a portion of the first balloon and the airway wall. Another step 314 comprises passing a medication through the infusion lumen and into the first balloon with a pressure sufficient to expel the medication through the one or more pores. Another step 316 comprises stopping passing the medication through the infusion lumen. Another step 318 comprises removing a portion of the fluid from the second balloon. Another step 320 comprises withdrawing the distal end of the catheter from the airway. Another step 322 comprises withdrawing the distal end of the guide wire from the airway.

While the guide wire and catheter have been described as separate devices, the guide wire can be integrated into the catheter to form a single device. For example, the guide wire can be integrated within the wall of the elongate tubular member. Alternatively, the guide wire can be disposed on the interior or exterior surface of the wall of the elongate tubular member.

The step of dilating the second balloon to a pressure sufficient to dilate the stricture can be accomplished by inflating the second balloon to a pressure in the range from about 2 ATM to about 12 ATM.

The steps of passing a fluid through the inflation lumen and into the second balloon with a pressure sufficient to inflate the second balloon and dilate the point of treatment within the airway and removing a portion of the fluid within the second balloon to a pressure sufficient to provide contact between a portion of the first balloon and the airway wall are both optional. For example, in an alternative, the steps of passing a fluid through the inflation lumen and into the second balloon with a pressure sufficient to inflate the second balloon and dilate the point of treatment within the airway and removing a portion of the fluid within the second balloon to a pressure sufficient to provide contact between a portion of the first balloon and the airway wall can be omitted and replaced with a step comprising passing a fluid through the inflation lumen and into the second balloon with a pressure sufficient to provide contact between a portion of the first balloon and the airway wall. It is considered advantageous to pass medication through the infusion lumen into the first balloon when the second balloon is in its inflated configuration (e.g., dilating the airway wall and/or contacting the airway wall) so that the medication can be infused within the airway wall.

In a further alternative, the step of passing a fluid through the inflation lumen and into the second balloon with a pressure sufficient to inflate the second balloon and dilate the point of treatment within the airway can be accomplished and the step of removing a portion of the fluid within the second balloon to a pressure sufficient to provide contact between a portion of the first balloon and the airway wall can be omitted. This advantageously allows for the infusion of medication while the second balloon is inflated to a pressure sufficient to dilate the point of treatment (e.g., a stricture).

The methods described herein can alternatively be used to treat one or more strictures within an airway. For example, the step of navigating the first distal end of the guide wire to a point of treatment within said airway comprises navigating the first distal end past the stricture within the airway. In addition, the step of navigating the second distal end of the catheter to a point of treatment within said airway comprises navigating the second distal end past the stricture within the airway. Furthermore, the step of passing a fluid through the inflation lumen and into the second balloon with a pressure sufficient to inflate the second balloon and dilate the point of treatment within the airway can comprise dilating a stricture within an airway.

While the step of navigating the first distal end of the guide wire past a stricture has been described, the first distal end of the guide wire can alternatively be navigated proximal to, or within, the stricture. In addition, while navigating the second distal end of the catheter past the stricture has been described, the distal end of the catheter can alternatively be navigated proximal to, or within, the stricture.

While various catheter configurations, steps, alternative steps, and optional steps have been described above with respect to treating tissue in an airway, these catheter configurations, steps, alternative steps, and optional steps can be included in, accomplished concurrently with, and/or accomplished in the alternative to, the methodologies, catheter configurations, steps, alternative steps, and/or optional steps described above/or below with respect to treating tissue in a bodily passage, sinus cavity, airway, and/or sinus passage.

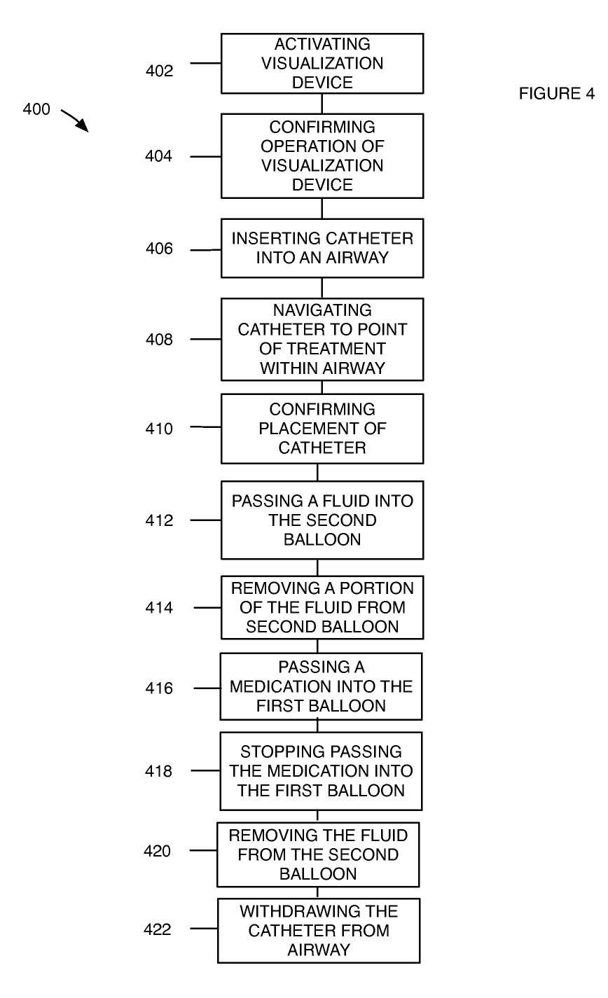

FIG. 4 is a flowchart representation of an exemplary method 400 of treating tissue within an airway defined by an airway wall. The method 400 is similar to that described above with respect to method 300, except as described below. An initial step 402 comprises activating a visualization device. Another step 404 comprises confirming operation of the visualization device. Another step 406 comprises inserting a catheter having a proximal end and a distal end into an airway such that the distal end of the catheter is disposed in the airway. Another step 408 comprises navigating the distal end of the catheter to a point of treatment within the airway. Another step 410 comprises confirming placement of the catheter. Another step 412 comprises passing a fluid through the inflation lumen and into the second balloon with a pressure sufficient to inflate the second balloon and dilate the point of treatment within the airway. Another step 414 comprises removing a portion of the fluid within the second balloon to a pressure sufficient to provide contact between a portion of the first balloon and the airway wall. Another step 416 comprises passing a medication through the infusion lumen and into the first balloon with a pressure sufficient to expel the medication through the one or more pores. Another step 418 comprises stopping passing the medication through the infusion lumen. Another step 420 comprises removing the fluid from the second balloon. Another step 422 comprises withdrawing the distal end of the catheter from the airway.

The step of activating a visualization device can be accomplished by activating a light source associated with a fiber optic disposed within the catheter and/or activating a camera disposed within the catheter. The fiber optic and/or camera can be disposed on an interior surface of a lumen of the catheter, embedded within the wall of the catheter, and/or disposed on an exterior surface of the catheter.

When a fiber optic is disposed within the catheter, the light source is adapted to be attached to the fiber optic, which is adapted to emit light radially and/or axially from the distal end of the fiber optic. When a camera is disposed within the catheter, the camera is adapted to capture images from the distal end of the catheter and/or from the circumference of the catheter at any point along the catheter length. The camera is adapted to be attached to a display and/or power source and can provide still and/or live footage to the display for review by the physician. Alternatively, multiple cameras can be used in conjunction with, or separate from, one another. The camera can comprise a wired or wireless camera that can transmit images to a display and/or a storage device.

The step of confirming operation of the visualization device can be accomplished by the physician verifying that light is being emitted from the fiber optic when the light source has been activated and/or the physician verifying that the camera is capturing images and displaying the images on a display when the camera has been activated.

The step of navigating the distal end of the catheter to a point of treatment can be accomplished transcutaneously and/or via images provided on a display. In an example, navigating the distal end of the catheter to a point of treatment using transcutaneous visualization can be accomplished by visualizing the intensity of the light being emitted from the fiber optic transcutaneously to determine if the intensity is indicative of proper navigation of the distal end of the catheter. In another example, navigating the distal end of the catheter to a point of treatment can be accomplished by viewing images provided on a display and determining if the images indicate proper navigation of the distal end of the catheter.

The step of confirming placement of the catheter can be accomplished transcutaneously and/or via images provided on a display. In an example, confirming proper placement of the distal end of the catheter at a point of treatment using transcutaneous visualization can be accomplished by visualizing the intensity of the light being emitted from the fiber optic transcutaneously to determine if the intensity is indicative of proper positioning of the distal end of the catheter. In another example, confirming proper placement of the distal end of the catheter at a point of treatment can be accomplished by viewing images provided on a display and determining if the images indicate proper positioning of the distal end of the catheter at a point of treatment. If the distal end is confirmed as being properly placed at a point of treatment, the step ends. However, if it is determined that placement of the distal end of the catheter is not proper, further navigation of the distal end of the catheter may be necessary.

While various catheter configurations, steps, alternative steps, and optional steps have been described above with respect to treating tissue in an airway, these catheter configurations, steps, alternative steps, and optional steps can be included in, accomplished concurrently with, and/or accomplished in the alternative to, the methodologies, catheter configurations, steps, alternative steps, and/or optional steps described above with respect to treating tissue in a bodily passage, sinus cavity, airway, and/or sinus passage.

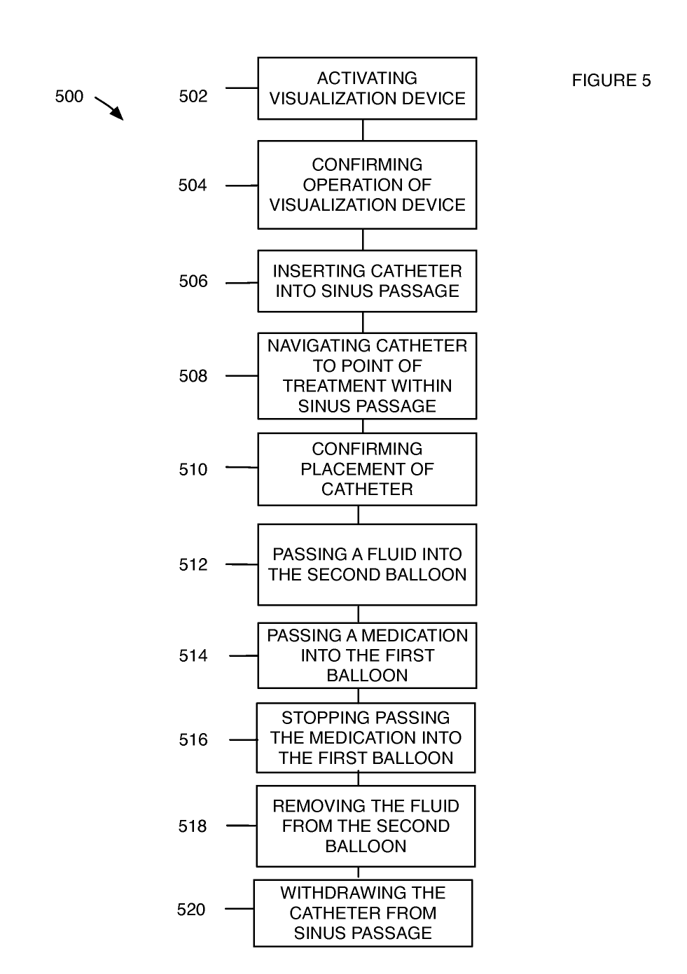

FIG. 5 is a flowchart representation of an exemplary method 500 of treating tissue within a sinus passage (e.g., eustachian tube) defined by a passage wall. The method 500 is similar to that described above with respect to method 400, except as described below. An initial step 502 comprises activating a visualization device. Another step 504 comprises confirming operation of the visualization device. Another step 506 comprises inserting a catheter having a proximal end and a distal end into a sinus passage such that the distal end of the catheter is disposed in the sinus passage. Another step 508 comprises navigating the distal end of the catheter to a point of treatment within the sinus passage. Another step 510 comprises confirming placement of the catheter. Another step 512 comprises passing a fluid through the inflation lumen and into the second balloon with a pressure sufficient to inflate the second balloon and provide contact between a portion of the first balloon and the passage wall. Another step 514 comprises passing a medication through the infusion lumen and into the first balloon with a pressure sufficient to expel the medication through the one or more pores. Another step 516 comprises stopping passing the medication through the infusion lumen. Another step 518 comprises removing the fluid from the second balloon. Another step 520 comprises withdrawing the distal end of the catheter from the sinus passage.

The step of passing a medication through the infusion lumen and into the first balloon with a pressure sufficient to expel the medication through the one or more pores can be accomplished by passing any suitable fluid, drug, and/or agent through the one or more pores. Examples of medications considered suitable include, but are not limited to, anti-inflammatories (e.g., steroids), antineoplastics (e.g., mitomycin), cytotoxics, adrenaline (e.g., epinephrine), antibiotics, antifungal agents, and/or anti-proliferatives (e.g., such as those used on coronary stents), or a combination thereof.

While various catheter configurations, steps, alternative steps, and optional steps have been described above with respect to treating tissue in a sinus passage, these catheter configurations, steps, alternative steps, and optional steps can be included in, accomplished concurrently with, and/or accomplished in the alternative to, the methodologies, catheter configurations, steps, alternative steps, and/or optional steps described above with respect to treating tissue in a bodily passage, sinus cavity, and/or airway.

The foregoing detailed description provides exemplary embodiments of the invention and includes the best mode for practicing the invention. The description and illustration of embodiments is intended only to provide examples of the invention, and not to limit the scope of the invention, or its protection, in any manner.

What is claimed is:

1. A method of treating tissue in an airway comprising the steps of:
inserting a catheter having a first proximal end and a first distal end into said airway such that the first distal end is disposed in said airway, the catheter comprising an elongate tubular member and a balloon, the elongate tubular member having a second proximal end, a second distal end, and an infusion port, the elongate tubular member defining an infusion lumen extending between the infusion port and the second distal end, the balloon disposed on the second distal end of the elongate tubular member and having a wall defining one or more pores extending through the wall of the balloon allowing fluid to pass through the wall of the balloon, the balloon in communication with the infusion lumen;
navigating the first distal end of the catheter to a point of treatment within said airway;
passing a medication through the infusion lumen and into the balloon with a pressure sufficient to expel the medication through the one or more pores;
stopping the step of passing a medication through the infusion lumen and into the balloon; and
withdrawing the first distal end of the catheter from said airway.

2. The method of claim 1, wherein an initial step comprises inserting a guide wire having a third proximal end and a third distal end into said airway such that the third distal end is disposed in said airway; and
wherein another step comprises navigating the third distal end of the guide wire to a point of treatment within said airway.

3. The method of claim 1, wherein the first distal end of the catheter comprises a camera configured to transmit images to a display.

4. The method of claim 1, wherein the medication expelled through the one or more pores comprises one of the group consisting of an anti-inflammatory agent, an antineoplastic agent, a cytotoxic agent, adrenaline, an antibiotic agent, an antifungal agent, and an anti-proliferative agent.

5. The method of claim 1, wherein the pressure sufficient to expel the medication through the one or more pores is between about 1 ATM and about 10 ATM.

6. A method of treating tissue in an airway comprising the steps of:
inserting a guide wire having a first proximal end and a first distal end into said airway such that the first distal end is disposed in said airway;
navigating the first distal end of the guide wire to a point of treatment within said airway;
advancing a catheter having a second proximal end and a second distal end over the guide wire such that the second distal end is disposed in said airway, the catheter comprising an elongate tubular member and a balloon, the elongate tubular member having a third proximal end, a third distal end, and an infusion port, the elongate tubular member defining an infusion lumen extending between the infusion port and the third distal end, the balloon disposed on the third distal end of the elongate tubular member and having a wall defining one or more pores extending through the wall of the balloon allowing fluid to pass through the wall of the balloon, the balloon in communication with the infusion lumen;
navigating the second distal end of the catheter to a point of treatment within said airway;
passing a medication through the infusion lumen and into the balloon with a pressure sufficient to expel the medication through the one or more pores;
stopping the step of passing a medication through the infusion lumen and into the balloon;
withdrawing the second distal end of the catheter from said airway; and
withdrawing the first distal end of the guide wire from said airway.

7. The method of claim 6, wherein the second distal end of the catheter comprises a camera configured to transmit images to a display.

8. The method of claim 6, wherein the medication expelled through the one or more pores comprises one of the group consisting of an anti-inflammatory agent, an antineoplastic agent, a cytotoxic agent, adrenaline, an antibiotic agent, an antifungal agent, and an anti-proliferative agent.

9. The method of claim 6, wherein the pressure sufficient to expel the medication through the one or more pores is between about 1 ATM and about 10 ATM.

10. A method of treating tissue in an airway defined by an airway wall, the method comprising the steps of:
inserting a catheter having a first proximal end and a first distal end into said airway such that the first distal end is disposed in said airway, the catheter comprising an elongate tubular member, a first balloon, and a second balloon, the elongate tubular member having a second proximal end, a second distal end, an inflation port, and an infusion port, the elongate tubular member defining an inflation lumen and an infusion lumen, the inflation lumen extending between the inflation port and the second distal end, the infusion lumen extending between the infusion port and the second distal end, the first balloon disposed on the second distal end of the elongate tubular member and having a wall defining one or more pores extending through the wall of the first balloon allowing fluid to pass through the wall of the first balloon, the first balloon in communication with the infusion lumen, the second balloon disposed within the first balloon and on the second distal end of the elongate tubular member, the second balloon moveable between a deflated configuration and an inflated configuration and in communication with the inflation lumen;
navigating the first distal end of the catheter to a point of treatment within said airway;
passing a fluid through the inflation lumen and into the second balloon with a pressure sufficient to inflate the second balloon and provide contact between a portion of the first balloon and said airway wall;
passing a medication through the infusion lumen and into the first balloon with a pressure sufficient to expel the medication through the one or more pores;
stopping the step of passing a medication through the infusion lumen and into the first balloon;
removing a portion of the fluid from the second balloon; and
withdrawing the first distal end of the catheter from said airway.

11. The method of claim 10, wherein an initial step comprises inserting a guide wire having a third proximal end and a third distal end into said airway such that the third distal end is disposed in said airway; and
wherein another step comprises navigating the third distal end of the guide wire to a point of treatment within said airway.

12. The method of claim 10, wherein the first distal end of the catheter comprises a camera configured to send images to a display.

13. The method of claim 10, wherein the point of treatment is a stricture within said airway.

14. The method of claim 13, wherein the step of passing a fluid through the inflation lumen and into the second balloon with a pressure sufficient to inflate the second balloon and provide contact between a portion of the first balloon and said airway wall comprises passing a fluid through the inflation lumen and into the second balloon with a pressure sufficient to inflate the second balloon and dilate the stricture.

15. The method of claim 14, wherein the pressure sufficient to inflate the second balloon and dilate the stricture is between about 2 ATM and about 12 ATM.

16. The method of claim 10, wherein the medication expelled through the one or more pores comprises one of the group consisting of an anti-inflammatory, antineoplastic, cytotoxic, adrenaline, antibiotic, antifungal agent, and anti-proliferative.

17. The method of claim 10, wherein the pressure sufficient to expel the medication through the one or more pores is between about 1 ATM and about 10 ATM.

18. The method of claim 10, further comprising the steps of:
inserting a guide wire having a second proximal end and a second distal end into said airway such that the second distal end is disposed in said airway;
navigating the second distal end of the guide wire to a point of treatment within said airway; and
wherein the step of navigating the first distal end of the catheter to a point of treatment within said airway comprises advancing the first distal end of the catheter over the guide wire such that the first distal end is disposed adjacent the point of treatment within said airway.

19. The method of claim 18, wherein the medication expelled through the one or more pores comprises one of the group consisting of an anti-inflammatory, antineoplastic, cytotoxic, adrenaline, antibiotic, antifungal agent, and anti-proliferative.

20. The method of claim 18, wherein the first distal end of the catheter comprises a camera configured to send images to a display.
